(19) United States
(12) Patent Application Publication
(10) Pub. No.: US 2007/0225355 A1
(43) Pub. Date: Sep. 27, 2007

(54) Dietary Compositions for Enhancing Metabolism and Reducing Reactive Oxygen Species

(75) Inventor: Cheng Liu, Richmond, CA (US)

Correspondence Address: CONWELL LLC, 2138 PRIEST BRIDGE COURT, SUITE 4, CROFTON, MD, 21114, US

(73) Assignee: Elixirin Corporation, Richmond, CA (US)

(21) Appl. No.: 11/571,321

(22) PCT Filed: Jun. 23, 2005
(86) PCT No.: PCT/US05/22383

§ 371 Date: Dec. 27, 2006

Related U.S. Application Data
(60) Provisional application No. 60/583,625, filed on Jun. 29, 2004.

(51) Int. Cl. A61K 31/385; C07D 339/04; C12N 1/02; C12N 1/26

(52) U.S. Cl. 514/440; 435/248; 435/261; 549/37

(57) ABSTRACT

This invention relates to dietary compositions and to methods for modifying cellular metabolism, metabolic production of reactive oxygen species and the resulting level of reactive oxygen species. The invention is drawn to a composition comprising a combination of carnitine, lipoic acid and polyphenol, which has the effect of enhancing metabolism and reducing oxygen species at the same time. When the composition is fed to older animals, the animals will have improved metabolism at the cellular level and a reduction of oxidative stress. The invention is also drawn to a method of oral administration of carnitine, lipoic acid and polyphenol to a mammalian host, at an effective dose necessary to affect enhanced metabolic processes and reduced oxygen species in animals including humans.

### CROSS-REFERENCE TO RELATED APPLICATIONS

The present application claims priority to U.S. Provisional Application No. 60/583,625, filed Jun. 29, 2004.

### BACKGROUND

1. Field of the Invention

This invention relates to dietary compositions and to methods for modifying cellular metabolism, the metabolic production of reactive oxygen species, and the resulting level of reactive oxygen species.

2. Background of Invention

Numerous lines of evidence suggest that reactive oxygen species are causing cellular damage through oxidation of the cell membrane, cellular proteins, and genomic DNA. There are multiple consequences of the damage caused by reactive oxygen species. Oxidation of the cell membrane reduces the level of non-saturated fatty acids, resulting in lower fluidity of the membrane, oxidation of cellular proteins compromise intracellular enzymatic activities and decrease metabolism, and oxidation of DNA increases the probability of gene mutations. These mechanisms of damage at the cellular level, either each alone or combined, will result in the lowered functions of tissues and organs, and eventually will have an adverse effect on health.

It is also well established that aged cells suffer from reduced level of mitochondrial metabolism (Shigenaga et al., Proc. Natl. Acad. Sci. USA, 91:10771-10778, 1994). The lower level of mitochondrial metabolism means lower production of ATP, the energy source of all cellular activities. Aged cells have decreased activity, resulting in lower function of an organ like heart, lung, or kidney, which are directly related to the health of the mammalian host.

U.S. Pat. No. 5,916,912 and WO 98/57627 (Ames et al.) disclose a dietary composition for enhancing metabolism and alleviating oxidative stress by oral administration to the host, an effective dosage of a carnitine, such as acetyl-L-carnitine, and an antioxidant, such as lipoic acid. U.S. Patent Application No. 2004/0044046 (Ames) discloses a method of stabilizing R-α-lipoic acid with nicotinamide and using said composition to treat oxidative stress.

There exists a need in the art for a dietary composition for enhancing metabolism and thus the energy of a subject and reducing reactive oxygen species in a single composition, without having to take separate compositions to achieve the same results. In addition, currently available supplements with a carnitine and lipoic acid combination does not provide enough anti-oxidation activity, which may result in a suboptimal health condition for the treated subject. This invention takes advantage of polyphenol, a class of natural anti-oxidants, which are found in red wine, dark chocolate, and green tea. Addition of polyphenol to the carnitine and lipoic acid combination solves the problem of insufficient anti-oxidant activity in the dietary complement described in the U.S. Pat. No. 5,916,912 and WO 98/57627 (Ames et al.). The combination of all three components can achieve a better health benefit than any of the previously-reported combinations.

### SUMMARY OF INVENTION

The present invention is drawn to a composition comprising a combination of carnitine, lipoic acid, and polyphenol which has the effect of enhancing metabolism and reducing reactive oxygen species at the same time. Polyphenol is a family of strong anti-oxidants that have been found in a variety of natural products such as green tea. A combination of these three components will have the cellular effect of enhancing ATP production while reducing reactive oxygen species. Carnitine and its derivatives are normal mitochondrial metabolites that facilitate transport of fatty acids to the mitochondria. Increasing carnitine levels is expected to enhance mitochondrial activity, therefore leading to higher levels of metabolism. Lipoic acid is a coenzyme in mitochondria that is involved in carbohydrate utilization necessary for the production of ATP and maintaining oxidative balance such glutathione levels.

In one embodiment of the invention, the combination of carnitine, lipoic acid and polypenol will increase the metabolic rate of aged cells of a mammalian host without increasing the production of reactive oxygen species. Therefore, when the combination is fed to older animals, the animals will have improved metabolism at the cellular level and a resulting reduction of oxidative stress. The animals will consequently experience reversal of several gross indicators of aging, including cognitive activity, and capacity of physical movement.

The present invention includes a method of oral administration of carnitine, lipoic acid and polypenol to a mammalian host at an effective dose necessary to affect enhanced metabolic processes and reduced reactive oxygen species in animals, including humans. The invention also includes a method of culturing a microorganism comprising culturing said microorganism in a medium comprising carnitine, lipoic acid and polyphenol.

### BRIEF DESCRIPTION OF THE FIGURES

FIG. 1 of 2 depicts spatial memory in rats subjected to a water maze test, after treatment with carnitine, lipoic acid, and/or polyphenol.

FIG. 2 of 2 depicts the measurement of oxidation levels of DNA in rats following a treatment with carnitine, lipoic acid, and/or polyphenol.

### DETAILED SUMMARY OF THE INVENTION

The present invention is drawn to a composition comprising a combination of carnitine, lipoic acid, and polyphenol in a subject. The composition may be used in a subject which includes animals such as farm animals, pets, and research animals, including, but not limited to bovine, ovine, porcine, equine, or avian animals; in feline or canine, or other animals such as ferrets, guinea pigs, rats, mice, llamas, alpacas, emus, water buffalo, bison, fish, reptiles, zoological specimens, and so forth. The composition may also be used in humans as a dietary supplement or clinically, as needed by a patient.

The present invention can also be used in the culture of microorganisms or animal cells as an additive for maximizing culture conditions in a laboratory or industrial setting (see Jay et al., U.S. Pat. No. 5,536,645, “Nutritive medium for the culture of microorganisms”).

The present invention is drawn to carnitine and its derivatives, intermediates and/or precursors, which are normal mitochondrial metabolites that facilitate transport of fatty acids to the mitochondria. Examples of carnitine and its derivatives, which are encompassed by the present invention include: acetyl-L-carnitine, mercapto acyl-carnitines, actetyl carnitine esters, mercapto carnitine esters, niconinoyl carnitine and derivatives, alkoxy-acyl derivatives of carnitine, alkoxy-acyl derivatives of carnitine, N-alkylamides of d(+)-carnitine, and compositions thereof.

Increasing carnitine levels in a host is expected to enhance mitochondrial activity, therefore leading to higher levels of metabolism. The effective dosage of carnitine of the invention is at least about 0.1 mg/kg host per day, at least about 1 mg/kg host per day, at least about 10 mg/kg host per day, at least about 50 mg/kg host per day, at least about 100 mg/kg host per day, at least about 200 mg/kg host per day, or at least about 250 mg/kg host per day. Dosages of the carnitine can also be administered in the range of at least about 0.1 mg/kg to at least about 1 g/kg, in the range of at least about 1 mg/kg to at least about 500 mg/kg more or in the range of at least about 4 mg/kg to at least about 50 mg/kg of body weight per day, although variations will necessarily occur depending on the formulation, host, and so forth. For use in microorganisms, at least about 0.005 mg/l to at least about 100 mg/l of carnitine can be used in the culture medium. It is understood that the dosages of carnitine may be greater or lesser than the dosages described herein, as an artisan in the field would appreciate and determine to be effective, and be within the scope of the present invention.

The present invention is also drawn to a composition comprising lipoic acid and its derivatives, intermediates and/or precursors. Alpha-lipoic acid is also known as thioctic acid, 1,2-dithiolane-3-pentanoic acid, 1,2-dithiolane-3-valeric acid and 6,8-thioctic acid. Alpha lipoinc acid can be present in two enantiomeric forms (R— and S—). The (R—) form of lipoic acid can present stability problems when stored, which can be solved by complexing R-α-lipoic acid with nicotinamide (U.S. Patent application 2004/0044046). Lipoic acid can be present in a reduced form, dihydrolipoate (DHLA) and is an excellent antioxidant capable of interacting with most forms of reactive oxygen species, recycling other antioxidants and additionally reducing oxidized disulfide groups in biological systems.

Lipoic acid is a coenzyme in mitochondria that is involved in carbohydrate utilization necessary for the production of ATP and maintaining oxidative balance such as intracellular glutathione levels. The effective dosage of lipoic acid of the invention is at least about 0.1 mg/kg host per day, at least about 1 mg/kg host per day, at least about 10 mg/kg host per day, at least about 50 mg/kg host per day, at least about 100 mg/kg host per day, at least about 200 mg/kg host per day, or at least about 250 mg/kg host per day. Dosages of the lipoic acid can also be administered in the range of at least about 0.1 mg/kg to at least about 1 g/kg, in the range of at least about 0.5 mg/kg to at least about 100 mg/kg more or in the range of at least about 1 mg/kg to at least about 10 mg/kg of body weight per day, although variations will necessarily occur depending on the formulation, host, and so forth. For use in microorganisms, at least about 0.005 mg/l to at least about 100 mg/l of lipoic acid can be used in the culture medium (U.S. Pat. No. 5,536,645). It is understood that the dosages of lipoic acid may be greater or lesser than the dosages described herein, as an artisan in the field would appreciate and determine the dosage needed to be effective, and be within the scope of the present invention.

The present invention is also drawn to a composition comprising polyphenol, derivatives of polyphenol and precursors thereof. Polyphenols such as (−)-epigallocatechin-3-gallate, derivatives, intermediates and precursors and the like are included in the invention. Polyphenol is a family of strong anti-oxidants that have been found in a variety of natural products such as green tea (Katiyar et al., J. Leuk. Biol. 69:719-726, 2001). The effective dosage of polyphenol of the invention is at least about 0.01 mg/kg host per day, at least about 0.1 mg/kg host per day, at least about 1 mg/kg host per day, at least about 10 mg/kg host per day, at least about 100 mg/kg host per day, at least about 200 mg/kg host per day, or at least about 250 mg/kg host per day. Dosages of the polyphenol can also be administered in the range of at least about 0.01 mg/kg to at least about 1 g/kg, in the range of at least about 0.05 mg/kg to at least about 500 mg/kg more or in the range of at least about 0.1 mg/kg to at least about 10 mg/kg of body weight per day, although variations will necessarily occur depending on the formulation, host, and so forth. For use in microorganisms, at least about 0.005 mg/l to at least about 100 mg/l of polyphenol can be used in the culture medium. It is understood that the dosages of polyphenol may be greater or lesser than the dosages described herein, as an artisan in the field would appreciate and determine to be effective, and be within the scope of the present invention.

A combination of these three components will have the cellular effect of enhancing ATP production while reducing reactive oxygen species. Measurements regarding parameters of aging in a host are well known in the art and include, but are not limited to, activities and behavior such as grooming, mental acuity and memory, sexual activity, dominance, physical strength, energy level, immune responses, cardiovascular symptoms, as well as physical appearances such as coat condition, wound repair, cellular and molecular lesions, muscle strength and tone, kidney appearance and function, and the like.

The effective dosage may be in a combination of carnitine in the range of at least about 0.1 mg/kg to at least about 1 g/kg, in the range of at least about 1 mg/kg to at least about 500 mg/kg more or in the range of at least about 4 mg/kg to at least about 50 mg/kg of body weight per day; lipoic acid can also be administered in the range of at least about 0.1 mg/kg to at least about 1 g/kg, in the range of at least about 0.5 mg/kg to at least about 100 mg/kg more or in the range of at least about 1 mg/kg to at least about 10 mg/kg of body weight per day; and polyphenol can also be administered in the range of at least about 0.01 mg/kg to at least about 1 g/kg, in the range of at least about 0.05 mg/kg to at least about 500 mg/kg more or in the range of at least about 0.1 mg/kg to at least about 10 mg/kg of body weight per day, although variations will necessarily occur depending on the formulation, host, and so forth. The effective dosage can be at least about 5 mg/kg host/day of carnitine, at least about 5 mg/kg host/day of lipoic acid, and at least about 5 mg/kg host/day of polyphenol for use in animals.

The dosage administered depends upon the age, health and weight of the subject, type of previous or concurrent treatment, if any, frequency of treatment, and the nature of the effect desired. The compositions of the invention can be administered by any means that achieve their intended purposes. Amounts and regimens for the administration of the composition according to the present invention can be determined readily by those with ordinary skill in the art. Administration of the composition of the present invention can also optionally be included with previous, concurrent, subsequent or adjunctive therapy in a clinical setting or as part of a dietary regimen.

In addition to the active compounds, a composition of the present invention can also contain suitable carriers acceptable for dietary use and/or pharmaceutical use comprising excipients and auxiliaries which facilitate processing of the active compounds into preparations which can be used pharmaceutically or as a dietary supplement. Suitable formulations for oral administration include hard or soft gelatin capsules, dragees, pills, tablets, including coated tablets, elixirs, suspensions, syrups or inhalations and controlled release forms thereof. Preferably, the preparations, particularly those preparations which can be administered orally and which can be used for the preferred type of administration, such as tablets; dragees, and capsules; softgels; blisters; functional foods, such as power bars, gums, candies, and the like; and functional drinks, such as soft drinks, juices, milks, soy drinks, power drinks, and the like. Drinks such as tea, herbal preparations, coffees and the like are also included in the invention.

Suitable excipients are, for example, fillers such as saccharide, lactose or sucrose, dextrose, sucralose (SPLENDA®), aspartame, saccharine, mannitol or sorbitol; cellulose preparations and/or calcium phosphates, such as tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone, and may also include preparations comprising natural honey or derivatives. If desired, disintegrating agents can be added such as the above-mentioned starches and also carboxymethyl starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl cellulose phthalate are used. Dyestuffs or pigments can be added to the tablets or dragee coatings, for example, for identification or in order to characterize combinations of active compound doses.

Other preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils or liquid paraffin In addition, stabilizers can be added.

Solid dosage forms in addition to those formulated for oral administration include rectal suppositories. The composition of the present invention can also be administered in the form of an implant when compounded with a biodegradable slow-release carrier. Suitable injectable solutions include intravenous subcutaneous and intramuscular injectable solutions. Alternatively, the composition of the invention may be administered in the form of an infusion solution or as a nasal inhalation or spray. Alternatively, the composition of the present invention can be formulated as a transdermal or transmucosal patch for continuous release of the active ingredient.

Possible preparations that can be used rectally include, for example, suppositories that consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

A formulation for systemic administration according to the invention can be formulated for enteral, parenteral or topical administration. Indeed, all three types of formulation can be used simultaneously to achieve systemic administration of the active ingredient.

Suitable formulations for parenteral administration include aqueous solutions of the active compounds in water-soluble form, for example, water-soluble salts. In addition, suspensions of the active compounds as appropriate oily injection suspensions can be administered. Suitable liphophilic solvents or vehicles include fatty oils, such as sesame oil, or synthetic fatty acid esters, such as ethyl oleate or triglycerides. Aqueous injection suspensions that can contain substances that increase the viscosity of the suspension include, for example, sodium carboxymethyl cellulose, sorbitol, and/or dextran. Optionally, the suspension can also contain stabilizers.

Suitable formulations for topical administration include creams, gels, jellies, mucilages, pastes and ointments.

The invention provides administratively convenient formulations of the compositions including dosage units incorporated into a variety of containers.

Convenient unit dosage containers include metered sprays, measured liquid containers, measured powdered containers and the like. The compositions can be combined and used in combination with other therapeutic or prophylactic agents. For example, the compounds may be advantageously used in conjunction with other antioxidants, free radical scavengers, and mixtures thereof, or other mixtures as known in the art, (e.g. Goodman & Gilman, The Pharmacological Basis of Therapeutics, 9^{th} Ed., 1996, McGraw-Hill). In another embodiment, the invention provides the subject compounds in the form of one or more pro-drugs, which can be metabolically converted to the subject compounds by the recipient host. A wide variety of pro-drug formulations are known in the art.

Compositions of the present invention are manufactured in a manner which is itself known, for example, by means of conventional mixing, granulating, dragee-making, dissolving, or lyophilizing processes. Thus, preparations for oral use can be obtained by combining the active compounds with solid excipients, optionally grinding the resulting mixture, and processing the mixture of granules, after adding suitable auxiliaries, if desired or necessary, to obtain tablets or dragee cores.

### EXAMPLES

### Example 1

Morris Water Spatial Memory in Rats

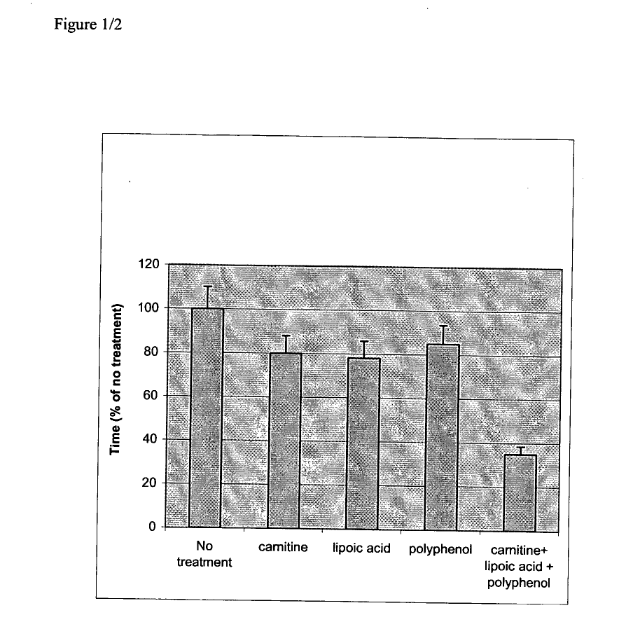

FIG. 1 of 2 depicts measurements of spatial memory in (Fischer 344 male rats) subjected to a water maze test, after treatment with carnitine, lipoic acid, and/or polyphenol. The rats that were treated with a combination of carnitine, lipoic acid, and polyphenol demonstrated greater than 50% faster mastery of the water maze than the rats treated with any of the components alone (approximately 38% faster for the combination over no treatment versus approximately 80%, 79% and 82% for carmitine, lipoic acid and polyphenol alone, respectively). The Morris maze task tests spatial memory by requiring rats to fmd a submerged platform in a pool of water using external visual cues (Morris, R. 1984; J. Neurosci. Methods, 11:47-60; Schenk, F. and Morris, R, Exp. Brain Res. 58:11-28, 1985). The rats in the experimental group (5 each) were fed either 0.5% (wt/vol) acetyl-L-carnitine in water, 1.0% (wt/vol) polyphenol in water; 0.2% (wt/wt) lipoic acid in AIN93M diet, or a combination of the above. Rats were acclimated for one week before experiments. Rats were fed for the above treatment for 7 weeks before tests. Trials (4 consecutive days, 4 trials per day) were conducted and the results averaged. The standard deviation is around 20%. Control rats were fed with water and AIN93M diet only.

Oxidation Levels of DNA

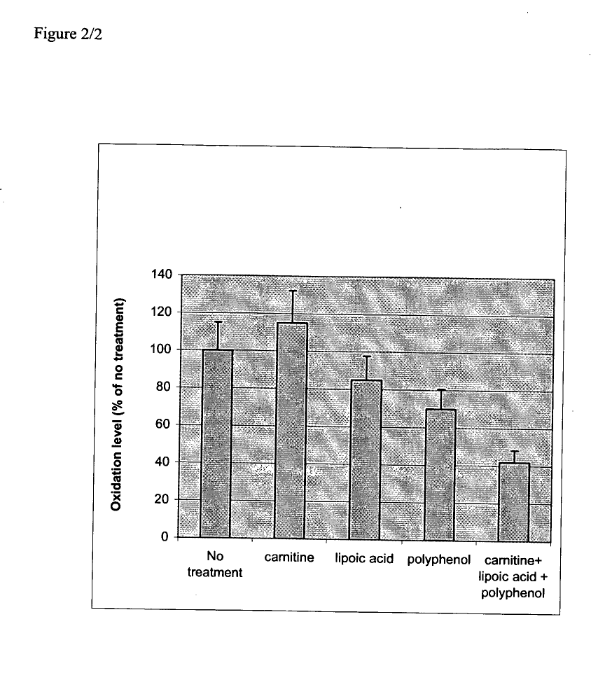

FIG. 2 of 2 demonstrates that the combination treatment of carnitine, lipoic acid, and/or polyphenol resulted in a lowering of oxidative stress in rats, as measured by the oxidation level of DNA. The combination treatment of carnitine, lipoic acid, and polyphenol resulted in an oxidation level of 41% compared with rats having no treatment. Rats treated with carnitine, lipoic acid, and polyphenol alone resulted in DNA oxidation levels of approximately 112%, 82%, and 65%, respectively. The rats in the experimental group (5 each) were fed either 0.5% (wt/vol) acetyl-L-camitine in water, 1.0% (wt/vol) polyphenol in water; 0.2% (wt/wt) lipoic acid in AIN93M diet, or a combination of the above. Rats were acclimated for one week before experiments. Rats were fed for the above treatment for 7 weeks before tests. Control rats were fed with water and AIN93M diet only. For assaying DNA oxidation, rats from each treatment group (5 animals per group) was anesthetized with ether and perfused with paraformaldehyde. The brain was removed and postfixed for paraffin sections. Sections of hippocampus were incubated with anti-8-hydroxy-2′-deoxyguanosine/8-hydroxyguanosine and visualized by using standard immunocytochemical methods. Quantification of oxidation was done as described in Liu, J K, et al., (Proc. Natl. Acad. Sci USA, 99:1356-2361, 2002). Values are a mean of 5 animals. The standard deviation is about 15%.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are incorporated by reference in their entirety.

1. A composition comprising carnitine, lipoic acid and polyphenol.

2. The composition of claim 1, wherein said composition comprises carnitine in the range of at least about 4 mg/kg to at least about 50 mg/kg subject per day, and lipoic acid in the range of at least about 1 mg/kg to at least about 10 mg/kg subject per day, and polyphenol in the range of at least about 0.1 mg/kg to at least about 1 mg/kg subject per day.

3. The composition of claim 2, wherein the carnitine is in the form of actyl-L-carnitine.

4. The composition of claim 2, wherein the lipoic acid is in the form of alpha-lipoic acid.

5. The composition of claim 2, wherein the polyphenol is in the form of (−)-epigallocatechin-3-gallate.

6. The composition of claim 1, wherein said composition comprises carnitine in the range of at least about 0.005 mg/l to at least about 100 mg/l, and lipoic acid in the range of 0.005 mg/l to at least about 100 mg/l, and polyphenol in the range of at least about 0.005 mg/l to at least about 100 mg/l.

7. The composition of claim 1, wherein said composition is in the form selected from the group consisting of hard gelatin capsule, soft gelatin capsule, dragee, pill, tablet, coated tablet, powder, elixir, suspension, syrup, inhaler, drink, candy, gum, and power bar.

8. The composition of claim 2, wherein said subject is selected from the group consisting of bovine, ovine, porcine, equine, avian, feline, canine, ferrets, guinea pigs, rats, mice, llamas, alpacas, emus, water buffalo, bison, fish, reptiles, and zoological specimens.

9. A method of modifying cellular metabolism comprising administering to a subject a composition comprising carnitine, lipoic acid and polyphenol.

10. The method of claim 9, wherein said composition comprises carnitine in the range of at least about 4 mg/kg to at least about 50 mg/kg subject per day, and lipoic acid in the range of at least about 1 mg/kg to at least about 10 mg/kg subject per day, and polyphenol in the range of at least about 0.1 mg/kg to at least about 1 mg/kg subject per day.

11. The method of claim 10, wherein the carnitine is in the form of actyl-L-carnitine.

12. The method of claim 10, wherein the lipoic acid is in the form of alpha-lipoic acid.

13. The method of claim 10, wherein the polyphenol is in the form of (−)-epigallocatechin-3-gallate.

14. The method of claim 10, wherein said subject is selected from the group consisting of bovine, ovine, porcine, equine, avian, feline, canine, ferrets, guinea pigs, rats, mice, llamas, alpacas, emus, water buffalo, bison, fish, reptiles, and zoological specimens.

15. A method of culturing a microorganism comprising culturing said microorganism in a medium comprising carnitine, lipoic acid and polyphenol.

16. The method of claim 15, wherein said medium comprises carnitine in the range of 0.005 mg/l to at least about 100 mg/l, and lipoic acid in the range of at least about 0.005 mg/l to at least about 100 mg/l, and polyphenol in the range of at least about 0.005 to at least about 100 mg/l.

17. The composition of claim 16, wherein the carnitine is in the form of actyl-L-carnitine.

18. The composition of claim 16, wherein the lipoic acid is in the form of alpha-lipoic acid.

19. The composition of claim 16, wherein the polyphenol is in the form of (−)-epigallocatechin-3-gallate.
